(19) United States
(12) Patent Application Publication
(10) Pub. No.: US 2024/0238013 A1
(43) Pub. Date: Jul. 18, 2024

(54) TEMPORARILY FIXABLE OSTEOSYNTHESIS DEVICE FOR VERTEBRAE, WITH A ROTATABLE FIXING ELEMENT

(71) Applicant: ORTHO HUB VENTURES UG (HAFTUNGSBESCHRÄNKT), Filderstadt (DE)

(72) Inventor: Frank HEUER, Filderstadt (DE)

(21) Appl. No.: 18/556,533

(22) PCT Filed: Mar. 30, 2022
(86) PCT No.: PCT/EP2022/058487

§ 371 Date: Oct. 20, 2023

(30) Foreign Application Priority Data
Apr. 22, 2021 (DE) 10 2021 002 120.7

(51) Int. Cl. A61B 17/70; A61B 17/00

(52) U.S. Cl. CPC A61B 17/7037; A61B 17/7032; A61B 2017/00858

(57) ABSTRACT

An osteosynthesis device for treating the spinal column is disclosed including a fork head which is U-shaped in a side view and has a through-opening and which, in the proximal direction, has two fork limbs with an internal thread, and in which a connecting rod can be received, and a ball-head receiving region is provided in the fork head in the distal direction in the through-opening, and a bone anchor is mounted pivotably therein, characterized in that there is provided on the fork head a transverse opening which communicates with the through-opening of the fork head, and a fixing element is mounted in this transverse opening so as to be rotatable about a transverse-opening axis, and the fixing element, with introduction of a torsional moment about the transverse-opening axis, clamps the head region of the bone anchor at a stable angle in the fork head.

### STATE OF THE ART

Various osteosynthesis devices for the care of the spine, such as for example pedicle screws, are known in the prior art. Such osteosynthesis devices are used to correct spinal misalignments or stabilize fractures by inserting and attaching the osteosynthesis devices into the vertebral bone and then connecting them together via longitudinal rods, or so-called connecting rods, in order to fix the vertebrae together in a desired position. In this case, the longitudinal rods are mounted on the osteosynthesis devices with the aid of locking elements, such as for example grub screws or other closure elements, and fixed in a non-slip manner. Pedicle screws are preferably used as osteosynthesis devices, which have a bone anchor that is pivotably mounted in at least one plane with a fork head and is angularly stable when the grub screw is fixed. Bone screws with a ball head are preferably used as bone anchors. Osteosynthesis devices with bone anchors and fork heads are usually mounted in such a way that the bone anchor in a proximal direction in the fork head is guided through the distal opening of the fork head. This only works if the outer diameter of the bone anchor shaft is smaller than the ball head diameter of the bone anchor and the outer diameter of the bone anchor shaft is smaller than the diameter of the distal opening of the fork head. Assembly is problematic if the outer diameter of the bone anchor shaft is larger than the opening diameter of the fork head and/or of the ball head diameter of the bone anchor.

A pedicle screw (DE102011053295A1) is known from the prior art, which can be temporarily locked and thus allows an extended range of applications in the care of spinal instabilities. This makes it possible to insert polyaxially movable pedicle screws into the vertebral bone in any orientation and then the user can temporarily clamp the angle between the fork head and the bone anchor. With a temporarily blocked polyaxiality, it is possible that corrective forces are induced on the bone anchor via the fork head and this has a direct effect on the setting and position of the vertebral bones. Without this temporary clamping, such corrective manoeuvres cannot be carried out or can only be carried out with difficulty. With such an arrangement, it is necessary for the thrust piece to be permanently clamped with the aid of an instrument and for the instrument to be attached to the screw at all times in order to maintain the compression force necessary for the temporary clamping. In some spinal correction manoeuvres, such as for example the correction of deformities and scoliosis, there is no room for such instruments. Therefore, it would be desirable to provide a screw implant that is independently able to maintain the temporary clamping once the instruments are removed.

From Application DE102018102173B3 a pedicle screw anchor is known in which the temporary compression force is permanently maintained by providing a detachable pin element. However, this construction requires a lever-like actuation of the thrust piece to generate the temporary clamping. This results in an increased combined compression and bending stress of the thrust piece, which leads to a mechanical limitation of the maximum temporary clamping effect. Furthermore, it is necessary for the thrust piece to be dimensioned in its material thickness in such a way that these loads do not have a destructive effect. As a result, such a pedicle screw is larger in its overall height than a regular pedicle screw. It is therefore desirable that the temporary clamping effect is not induced via the thrust piece itself, but acts directly on the bone anchor head region. Thus, the thrust piece is mechanically decoupled during the temporary clamping and the osteosynthesis device can provide reserves with regard to the maximum clamping effect.

Furthermore, it can be seen that none of these temporarily clampable pedicle screw designs is able to accommodate screw shafts coming from the distal direction. They can only be used with bone anchors in which the outer diameter of the bone anchor shaft is smaller than the diameter of the distal opening of the fork head.

An object of the invention is to provide a temporarily clampable osteosynthesis device, in particular a pedicle screw, which allows a bone anchor to be mounted coming from the distal direction and that the thrust piece is not loaded when the temporary clamping is actuated, but is only subjected to a compressive force when the osteosynthesis device is finally locked with the connecting rod and the closure element. On the one hand, this results in a modularity of the screw system, which has advantages with regard to the reduced capital commitment of the user, and an overall larger screw portfolio can be offered. On the other hand, the force-flow distribution in the case of the temporary clamping without the mechanical involvement of the thrust piece results in a significant gain in stability compared to previous designs, and the pedicle screw can be made smaller in its overall height. At the same time, the design according to the invention is intended to maintain the temporarily generated clamping independently, even if all instruments have been removed.

### DESCRIPTION OF THE INVENTION

The invention relates to an osteosynthesis device, in particular a polyaxial pedicle screw, consisting of a bone anchor having a head, a fork head that is U-shaped in a side view, a thrust piece located therein, and an inner-lying fixing element guided in a transverse opening, wherein the fixing element is suitable for temporarily clamping the bone anchor head region in all degrees of freedom by rotation about the transverse opening axis, without thereby a thrust piece being necessary or, if present, not being loaded.

In the preferred embodiment, it is possible to induce the clamping effect indirectly via a positioning element or directly via the fixing element itself without the connecting rod or grub screw being present. Since this is not a final clamping with an inserted connecting rod, this type of clamping is called temporary clamping. With the temporary clamping, it is possible for the user to convert a polyaxial screw into a monoaxial screw in a desired angular position during surgery. This means that all rotational degrees of freedom of a polyaxial screw are temporarily blocked. The screw behaves monoaxially. This allows the user to manipulate the vertebra to be treated both translationally and rotationally until he inserts a connecting rod in the desired end position and fixes it with the grub screw. Such correction manoeuvres are not possible with a polyaxial screw, since a correction manoeuvre initiated on the outside of the patient side results in a free movement of the polyaxial ball joint and is therefore not transmitted to the vertebra. This only works with deactivated rotational degrees of freedom in the ball joint, i.e. it is temporarily clamped.

Regardless of the temporary clamping, after implantation of the osteosynthesis device in the bone, a connecting rod must be inserted and the osteosynthesis device finally fixed in all degrees of freedom with the aid of a closure element. This is done by screwing the closure element tightly.

When the closure element is tightly fastened, an axial compression force is transmitted from the closure element on the connecting rod, and the latter presses on the rod supporting points of the thrust piece and generates a minimal relative movement of the thrust piece further distally, so that the bone anchor is clamped angularly stable in the ball seat. Optimally, two or more osteosynthesis devices are connected to one another with the aid of a connecting rod.

The fork head is here configured in such a way that a connecting rod can be inserted and fixed to the fork head with a locking element. As already mentioned, this achieves an angularly stable clamping between the bone anchor head region and the fork head. The angularly stable clamping with the aid of the locking element and the angularly stable clamping with the aid of the fixing element work independently of one another in the structure according to the invention. They can be activated separately but also in combination.

In a preferred embodiment of the osteosynthesis device, the fork head has a through opening and, in the proximal direction, forms two fork legs with an inner-lying thread for a locking element. In the distal direction, a ball head receiving region is provided in the through opening, in which a bone anchor is pivotably mounted.

Bone screws that can be screwed to a bone are preferably used as bone anchors. However, hooks, blade-like anchors, clamps, nails and differently designed bone anchors can also be used. The essential features of the bone anchor are a ball-like head, a neck region, and a region that can be anchored or attached to the bone. This patent application is intended to deal mainly with the fork head, and bone anchors are intended to be understood to mean all conceivable elements that can be connected to a bone.

An essential feature of a preferred embodiment is that the centre of the inner-lying thread and the centre of the ball head receiving region define the position and orientation of the central axis. An axial opening for the positioning element is provided laterally and at a distance that is greater than the diameter of a locking element. The axial opening is arranged mainly parallel to the central axis. The positioning element can thus be driven from the same direction as the locking element with the aid of instruments. In the axial opening, the positioning element is guided in a length-adjustable manner. In addition to openings completely enclosed by material, the term axial opening also refers to partial openings or partial cutouts such as, for example, c-shaped cutouts transversely to the central axis.

An additional opening, a transverse opening, is provided transversely to the axial opening and the central axis. The transverse opening establishes a connection between the axial opening and the through opening of the fork head. In this transverse opening, the fixing element is guided rotatably about the transverse opening axis. With the induction of a torsional moment about the transverse opening axis, the fixing element clamps the head region of the bone anchor in an angularly stable manner in the fork head. The transverse opening or the axis of the transverse opening is arranged approximately orthogonally to the central axis and also orthogonal to the axial opening.

The temporary clamping or compression force is preferably generated by a positioning element, which is guided in the axial opening, by adjusting the positioning element. The compression force of the positioning element is transmitted or deflected here to the fixing element, so that the fixing element rotates about the transverse opening axis and a torsional moment is thereby generated, which leads to the temporary clamping of the bone anchor head region in the fork head.

The temporary clamping effect can be significantly increased if a lever is provided on the fixing element. An actuation of the lever forces a rotation of the fixing element and thus triggers the temporary clamping. For the structure, it is necessary for the fixing element to protrude laterally beyond the walls of the fork head, so that the lever is firmly connected to the fixing element. In an alternative embodiment, the positioning element can now grasp the lever end in order to apply a force to the lever permanently and without additional instruments.

Alternatively, it is also conceivable that the temporary clamping or the induction of a compression force can take place via the fixing element itself. For this purpose, it would be necessary, for example, for the fixing element and the transverse opening to be in engagement with one another with the aid of a threaded section, thus enabling a longitudinal adjustment. Through this length adjustment the fixing element displaces itself along the transverse opening axis. If a change in diameter is provided along the fixing element, a force closure between the fixing element and the head region of the bone anchor can be achieved here. Optimally, the fork head provides at least a lateral bulge or material thickening in order to receive therein the axial opening, transverse opening and the elements necessary for actuation (positioning and fixing element). In the alternative embodiment with an additional lever, it is advantageous if a bulge is provided on each of both fork head legs (11, 12).

In a preferred embodiment, the fork head has a thrust piece which has a distally directed contact region towards the bone anchor head region and a proximally directed stave bearing. In the region of the bone anchor head region, a lateral opening or a partial cutout is provided, in which the fixing element is arranged in a freely movable manner. This allows the fixing element to be activated without the thrust piece being loaded.

In a preferred embodiment, the fork head may be mounted with bone anchors coming from the distal direction. Due to this advantageous arrangement of the components, the bone anchors can be mounted relatively easily with the fork head by placing or pressing them thereon. The bone anchor can also be removed again by means of an aid, such as for example a release instrument. The osteosynthesis device according to the invention can thus be configured modularly by the user and assembled in the operating room at a later time than that of production. This makes it possible, for example, for the bone anchor to be anchored or screwed individually into the bone first, and then for the fork head to be attached to the already implanted bone anchor. This has the great advantage that after the implantation of the bone anchor, the surgeon has significantly more space and a better view in the surgical field compared to the otherwise fully implanted pedicle screws.

The advantage is that, on the one hand, larger bone anchors, i.e. bone anchors with a larger outer diameter, than the distal inner diameter of the fork head can be mounted. On the other hand, the bone anchor portfolio can be minimized because the user can combine the fork head and bone anchor during surgery instead of resorting to a prefabricated oversized portfolio. Such a portfolio must be in stock with the user and thus significantly more capital is tied up than with the modular version according to the invention would require.

For a modular design of the osteosynthesis device, it is advantageous for the thrust piece to have open slots in the distal direction, and for at least three resilient arms to be designed on the head receiving region as a result. The resilient arms can deflect radially outwards and thus enclose the bone anchor head region. As a result, a bone anchor coming from the distal direction can be clipped into the thrust piece. The thrust piece forms a cone at least in sections at the distal end of the outer side. At least one inner cone section is defined in the fork head at the level of the ball receiving region, which leads congruently to the cone of the thrust piece and, when the locking element is actuated, to the angularly stable clamping of the bone anchor head region with the fork head. Here, too, it is essential for the thrust piece to provide a passage opening for the fixing element, so that the thrust piece is not loaded when the temporary clamping is activated.

At the proximal fork head portion there is provided a circumferential groove having a hook-like profile that provides a rear grip for an instrument. Representatively, differently designed groove profiles or other retaining features, such as openings, are conceivable, which provide a rear grip for an instrument.

At the proximal end of the fork head, there may be further and detachable portions with a threaded region that allow repositioning the connecting rod. It is also conceivable that a sleeve-like access formed by two longer legs is provided, as is used for minimally invasive access. The detachable leg extensions may optionally be connected to one another at the proximal end. By detachable connection are meant, for example, predetermined breaking points which are suitable for removing the extensions after the connecting rod has been finally fixed.

All metal alloys that are known and accepted as orthopaedic implant material are suitable for use as a material. These include, for example, titanium, cobalt-chromium and stainless steel alloys. If the conventional production of the fork head and of the locking ring is not possible or only possible with the highest technological effort, additive manufacturing is the means of choice. Additive manufacturing of metal alloys, also known as 3D printing, uses the laser or electron beam melting process.

Further features, advantages and details of the invention emerge from the attached patent claims, the drawings and the following description of preferred embodiments of the osteosynthesis device according to the invention.

### BRIEF DESCRIPTION OF THE DRAWINGS SHOWING

FIG. 1 an oblique view of the osteosynthesis device according to the invention with the pre-location of the spatial relationships,

FIG. 2 the fully implanted osteosynthesis device in an oblique view,

FIG. 3a an oblique view of the osteosynthesis device according to the invention, and

FIG. 3b an exploded view of the osteosynthesis device according to the invention consisting of a fork head, a fixing element, positioning element, bone anchor and thrust piece,

FIG. 4 a side view of the mounted osteosynthesis device according to the invention with the associated sectional view,

FIG. 5a, b two different positions S1, S2 of the positioning element.

FIG. 6 shows an exploded view of an alternative embodiment in which the fixing element has a lever.

FIG. 7 presents a side view of the mounted osteosynthesis device according to the invention from FIG. 6 with a corresponding sectional view.

FIGS. 8a and 8b illustrate the operation of the lever.

FIGS. 9a, b show an alternative embodiment in which levers are actuated with a positioning element.

FIG. 10 depicts a side view with a corresponding sectional view of the embodiment of FIGS. 9a and 9b.

### DESCRIPTION OF THE PREFERRED EMBODIMENTS

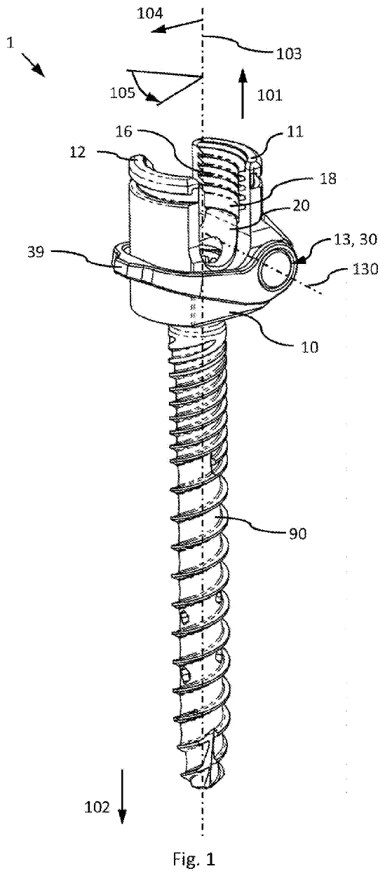

An osteosynthesis device (1) for treating the spine is described, wherein more than one osteosynthesis device (1) is used to connect one or more vertebrae to one another with the aid of connecting rods (50) and thus to stabilize the spine. For the osteosynthesis device (1), in particular for the fork head (10), coordinate references to be associated spatially are define, such as the proximal direction (101), the distal direction (102), which extend along a central axis (103). Starting from the central axis (103) outwards, the radial extent (104) is defined and the circumferential extent (105) is defined by a constant radius and a variable circumferential angle (FIG. 1).

FIG. 1 shows an embodiment of the osteosynthesis device (1) according to the invention for treating the spine, consisting of a fork head (10) which is U-shaped in a side view and has a through opening (18), and the fork head (10) has two fork legs (11, 12) in the proximal direction (101) with an inner-lying thread (16), and a connecting rod (50) can be received therein, and a ball head receiving region (19) is provided in the fork head (10) in the distal direction (102) in the through opening (18), and a bone anchor (90) is pivotably mounted therein. In the fork head (10) a transverse opening (13) is provided, which communicates with the through opening of the fork head (18).

In this transverse opening (13) a fixing element (30) is guided so as to be rotatable about an axis (130). With the induction of a torsional moment about the transverse opening axis (130), the fixing element (30) clamps the head region (91) of the bone anchor (90) in an angularly stable manner in the fork head (10). When the torsional moment at the fixing element (30) is released, the head region of the bone anchor (91) in the fork head (10) becomes movable again.

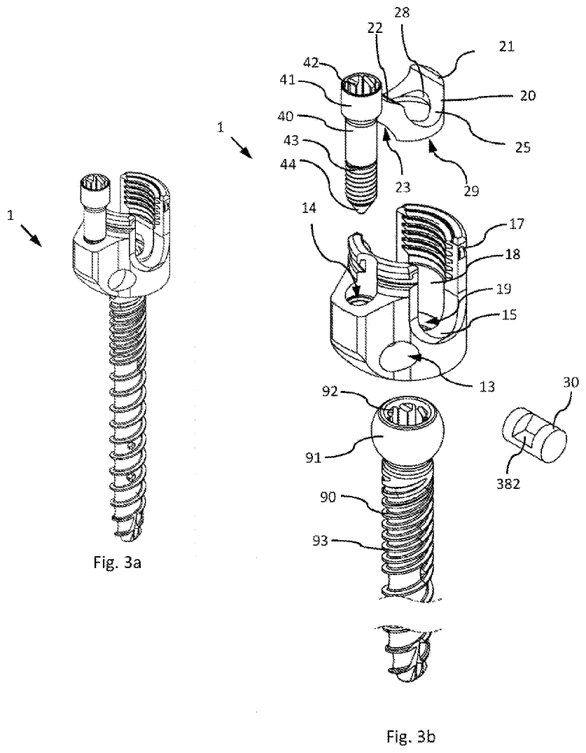

FIGS. 1, 3a and 3b also illustrate that the fork head (10) of the osteosynthesis device (1) has a thrust piece (20) and that the thrust piece has a through opening (28), a distally directed contact region towards the bone anchor head region (29), a proximally directed stave bearing (25), which is delimited by two legs (21, 22) and has a lateral opening or partial cutout (23) in the region of the bone anchor head region (29), in which the fixing element (30) is arranged in a freely movable manner. If a connecting rod (50) is inserted into the U-shaped fork opening (15), the connecting rod (50) is in direct contact with the stave bearing of the thrust piece (25). If the locking element (60) is now fixed with the fork head (10), a compression force is transmitted from the locking element (60) to the connecting rod (50), and from the latter to the thrust piece (20, 25) and from the thrust piece (20) to the bone anchor head region (29, 91), and the bone anchor (90) against the distal ball seat region (19). This induced compression force then leads to the clamping of the polyaxiality.

The bone anchor (90) preferably has a head region (91) with a tool attachment point (92) located therein and has a bone thread (93) in the distal direction (102).

FIG. 3b shows a preferred structure of the fixing element (30). It is depicted here that the fixing element (30) is preferably designed in the main overall shape as a round rod or pin and is rotatably mounted in a concentric transverse opening (13). Alternative embodiments of the fixing element (20), which are not shown here, are also conceivable, such as, for example, in the case of the fixing element in a sectional view transverse to the transverse opening axis (130), at least one section being designed as a triangle, quadrilateral or polygon, or else as a triangle or polygon with transitional roundings, or being provided as a solid round with lateral flattenings, which serve to ensure that a rotation of the fixing element (30) causes a clamping at the bone anchor head region (91). It is also conceivable for the fixing element (30) to change its geometry along the transverse opening axis (130) to provide engagement features for captive elements.

In the embodiment shown in FIG. 3b, the fork head (10) has an axial opening (14), in which a positioning element (40) is guided in the axial opening (14) and a compression force is generated by adjusting the positioning element (40), which compression force is transmitted directly to the fixing element (30). As a result, a torsional moment is generated at the fixing element (30) about the transverse opening axis (130), which leads to the temporary clamping of the bone anchor head region (91) in the fork head (10). It is advantageous if the axial opening (14) for the positioning element (40) is arranged parallel but at a distance from the central axis (103). The osteosynthesis device (1) preferably has a pin-shaped positioning element (40), which has a tool attachment point (42) on the head (41) in the proximal direction (101) (FIG. 3a, b). The positioning element (40) is designed to be detachable and, if necessary, can also be removed. With the positioning element (40) removed and the fixing element (30) not temporarily clamped, this osteosynthesis device (1) behaves like a polyaxial pedicle screw.

FIG. 3b also shows, if an axial opening (14) is provided in a fork head leg (11, 12), that it is advantageous for the fork head (10) to have a lateral material bead, which merges into one of the legs (11 or 12), so that the transverse opening (13) as well as the axial opening (14) can be produced at all.

It can also be inferred from FIG. 3b that the fork head (10) has projections, openings, grooves, bars, profiles or other features that are suitable for gripping, engaging or engaging from behind with an instrument. If the positioning element (40) is part of an instrument, this instrument binding feature (17) can be used to induce a tensile force on the fork head (10), which acts as an antagonist to the induction of a compression force via the positioning element (40).

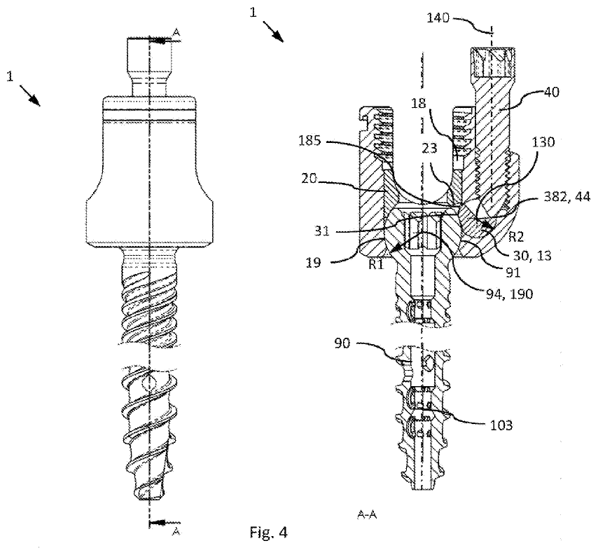

FIG. 4 shows the osteosynthesis device (1) according to the invention in a side view and in section. It can be seen that the centre of the inner-lying thread (16) and the centre of the ball head receiving region (190) define a central axis (103), and this central axis (103) does not intersect with the transverse opening axis (130). The transverse opening axis (130) is arranged approximately orthogonally and at a distance from the central axis (103). The fixing element (30) protrudes at least in sections into the through opening (18) of the fork head (10) (185) and abuts against the ball head region (91, 31) of the bone anchor (90). The transverse opening (13) communicates with the through opening of the fork head (18) via a wall cutout (185). Through this wall cutout (185), the fixing element (30) has direct access to the bone anchor head region (91). Optimally, the contact region of the fixing element (31) is located exactly at this point of the wall cutout (185).

Furthermore, it is advantageous if the contact point (31) for inducing the temporary clamping in the proximal direction (101) lies above the equator (94) of the bone anchor head region (91) or the centre (190) of the ball head receiving region in the fork head (19). As a result it is achieved, when the compression force is induced by the fixing element (30), the bone anchor head region (91) is not only pushed in the opposite direction or against the opposite inner wall of the fork head (18), but is also partially forced into the ball seat (19). Thus, the bone anchor (90) is centred in the fork head (10) in the ball seat (19) under the action of force. As a result, the compression force is directed toward the midpoint (190) of the ball-like bone anchor head region (91), so that a similarly large contact region is present between the fixing element and the bone anchor head region (31, 91) even when the bone anchor (90) is pivoted.

The radially inwardly directed contact region (31) of the fixing element (30) which is responsible for the temporary clamping, abuts directly against the head region (91) of the bone anchor (90). It is advantageous if this contact region (31) is concavely shaped at least in sections in a side view or approximates at least a section of the outer surface of the bone anchor head region (91). For an optimized clamping effect, it is advantageous if the radially inwardly directed contact region (31) of the fixing element (30) has an increased roughness, notches or teeth (FIG. 4). It is advantageous for the production if the radially inwardly directed contact region is configured as a cutout of the otherwise pin-like fixing element. In order for the pin-like fixing element (30) to be able to exert a clamping effect, it is advantageous if the head region of the bone anchor (91) is defined by a radius R1 and the fixing element (30) at its thickest point by a radius R2, and the shortest distance between the central axis (103) and the transverse opening axis (130) is smaller than the sum of the radii R1 and R2. As a result, there is a calculated material overlap of the bone anchor head region with the fixing element. The contact region (31) cut out in the fixing element provides the corresponding clearance to the bone anchor head region. A rotation of the fixing element (30) about the transverse opening axis (130) forces a material overlap, which then results in the clamping of the bone anchor head region (91) in the ball seat of the fork head (19). Alternatively, this can also be achieved via an eccentric in sections as part of the fixing element (30).

FIG. 4 also shows that a positioning element (40) is provided. By adjusting the positioning element (40), a compression force can be generated, which is transmitted or deflected to the fixing element (30), and a torsional moment about the transverse opening axis (130) is thereby generated, which leads to the temporary clamping of the bone anchor head region (91) in the fork head (10).

In this case, the positioning element (40) is guided in the axial opening (14) in a longitudinally adjusted manner. If the positioning element (40) is part of the implant, it is advantageous if the axial opening (14) for the positioning element (40) has an inner thread at least in sections. Being engaged with this, it is necessary for the positioning element (40) itself to have a thread (43) at least in sections. As a result, it is possible for the induced compression force to be maintained when the positioning element (40) is screwed in or fastened. If the positioning element (40) is part of an instrument, the positioning element (40) itself does not have to have a thread, since the compression force to be induced is generated by the instrument.

For the deflection of the compression force from the positioning element (40) to the fixing element (30), it is advantageous if the fixing element (30) has at least a contact surface (382) which is in direct contact with a distal contact region (44) of the positioning element (40), and this contact (382, 44) is configured in such a way that a compression force along the positioning element axis (140) is diverted into a rotation about the transverse opening axis (130). In this case, the contact surface (382) of the fixing element (30) acts as an integrated lever (FIG. 4). It is therefore important that the axis of the positioning element (140) is at a distance from the transverse opening axis (130). Furthermore, in this embodiment it is provided for the distal contact region (44) of the positioning element (40) to be convex, at least in sections, in order to ensure a tangential guidance or constant contact during induced rotation of the fixing element.

In FIG. 4 the essential characteristic feature can also be seen, namely that when the bone anchor head region (91) is temporarily clamped solely by the fixing element (30), the thrust piece (20) is always unloaded. A force applied to the thrust piece only by inserting and fixing a connecting rod (50, 60). As a result, the fork head and the thrust piece can absorb a higher mechanical load, and the thrust piece can be provided with significantly less material than comparable concepts, which in turn has a positive effect on the overall height of the osteosynthesis device (1). In osteosynthesis devices (1) for the spine, the smallest possible overall height is important in order to best adapt to the anatomical conditions of the patient.

The osteosynthesis device (1) is configured in such a way that a connecting rod (50) can be inserted and by means of a locking element (60) can be fixed to the fork head (10), thereby achieving an angularly stable clamping between the bone anchor head region (91) and the fork head (10), and the angularly stable clamping with the aid of the locking element (60) and the angularly stable clamping with the aid of the fixing element (30) can be activated independently of one another and can also be combined with one another.

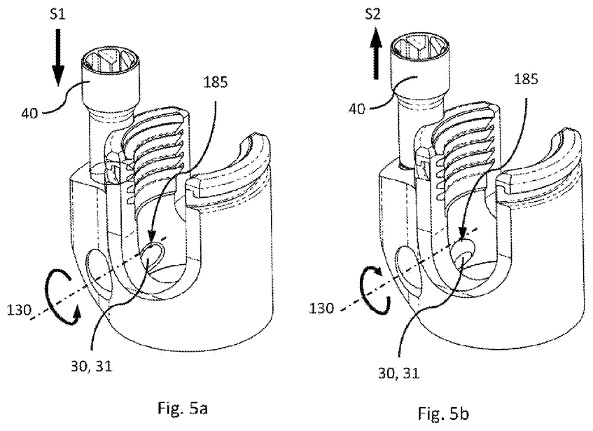

From FIGS. 5a and 5b it can be inferred that the fixing element (30) can assume a position S1 about the transverse opening axis (130), in which a compression force is transmitted on the bone anchor (90), so that bone anchor (90) is held angularly stable in the ball seat (19) and the fixing element (30) can assume a second position S2, in which the bone anchor (90) is movably held in the ball seat (19). These positions of the fixing element (30) can be adjusted by adjusting the positioning element (40). This can be seen from the different rotational positions of the fixing element (30, 31) in the wall cutout (185).

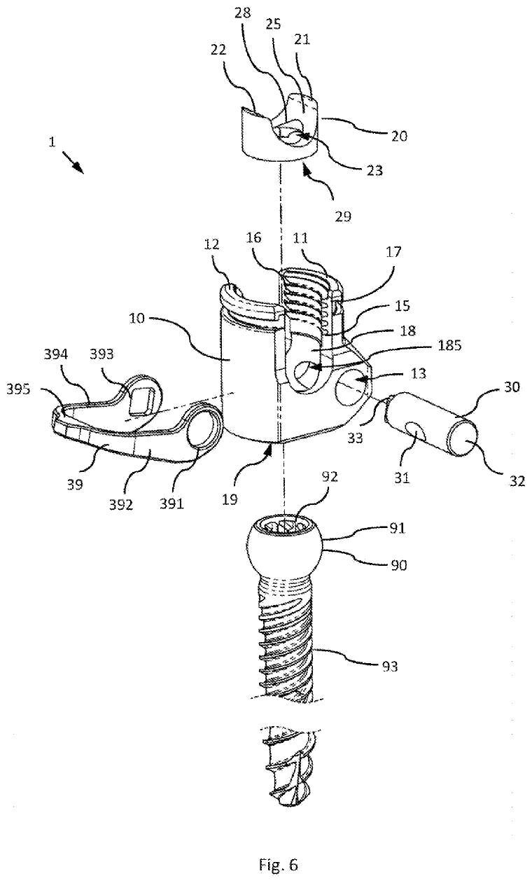

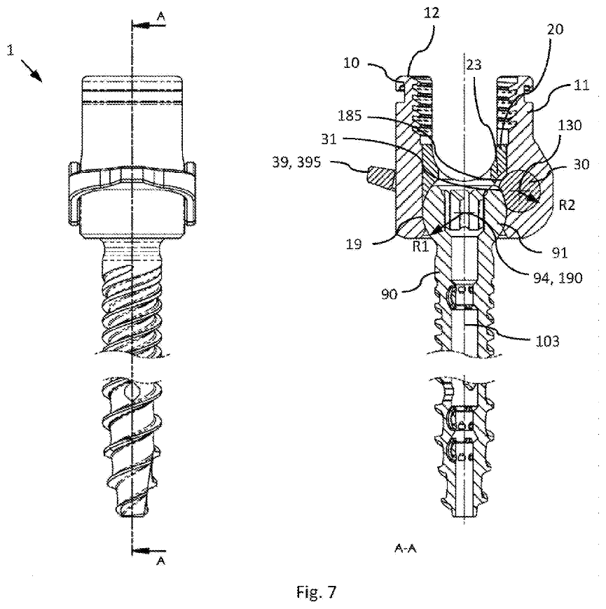

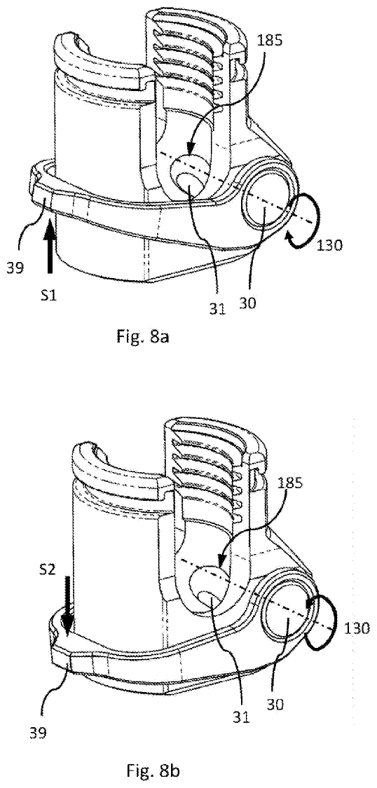

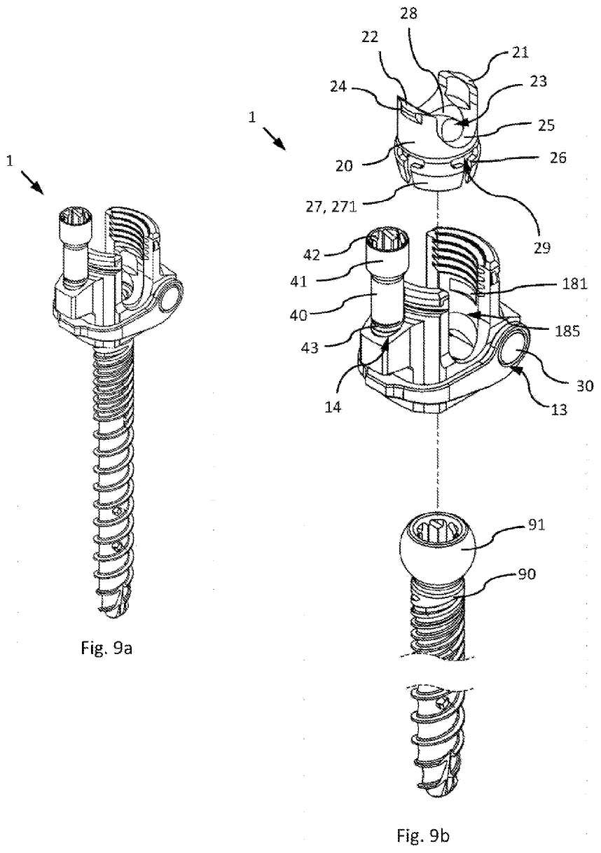

FIG. 6 and FIG. 7 show an alternative embodiment of the osteosynthesis device in which the clamping force can be increased by an additional lever (39). Here, it is advantageous if the fixing element (30) extends (32, 33) beyond at least one lateral wall of the fork head (10) and the lever (39) is attached thereto. An actuation of the lever (39) causes a rotation of the fixing element (30) and thus forces the temporary clamping of the bone anchor head region (91) in the fork head (10). The deflection of the lever is preferably induced at a region (395) which is at the greatest possible distance from the transverse opening (13) in order to maximize the lever effect, i.e. the force amplification. Optimally, the fixing element (30) has two ends (32, 33) which protrude beyond the lateral wall of the fork head. The lever preferably has two legs (392, 394), which are connected to the lever or joined to one another (391, 393) at the ends of the fixing element (32, 33). It is advantageous if the joining points additionally contain a form fit (393), so that a higher transmission of loads is possible. Optimally, the lever is arranged outside the fork head (10) so that it can be actuated with suitable positioning means such as the positioning element (40, FIGS. 9a, 9b) or an instrument not shown here. The actuation or the shift between two positions of the positioning with the clamping effect being adjusted is depicted in FIGS. 9a and 9b.

FIGS. 9a and 9b show a further preferred embodiment of the osteosynthesis device (1) according to the invention. Here, the thrust piece (20) has open slots (26) in the distal direction (102). As a result, at least three resilient arms (27) are formed on the head receiving region (29), wherein the resilient arms (27) describe a cone (271) on their outer side at least in sections, and the bone anchor (90) can be fitted into the fork head (10) coming from the distal direction (102). As a result, bone anchors (90) with a larger outer diameter can be mounted with the fork head (10).

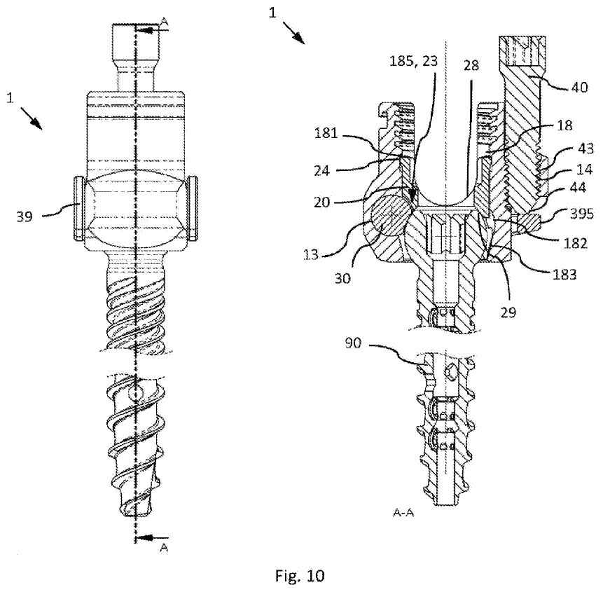

So that the thrust piece can be optimally clamped within the fork head (10), at least one inner cone section (183) is defined in the ball receiving region (19), which is provided congruently with the cone (271) of the thrust piece (20) and, when the locking element (60) is actuated, leads to the angularly stable clamping of the bone anchor head region (91) with the fork head (10) (FIGS. 9a, b and FIG. 10). Here, too, it is necessary for the thrust piece to have a lateral opening or an at least partial cutout (23) through which the fixing element (30) is guided and can move therein. This ensures that the thrust piece (20) is not loaded when the temporary clamping is activated.

FIGS. 9a, 9b also illustrate an alternative embodiment in which the lever (39) can be actuated by a positioning element (40). This structure is suitable for maintaining the clamping effect through the positioning means (40) without instruments.

It can be inferred in FIG. 10 that the fork head legs (11, 12) each provide a supporting surface (181) with an undercut, which is effective in the proximal direction (101), and the thrust piece (20) has radially outwardly directed projections (24) at the proximal end, the projections (24) being designed to be resilient radially inwards, so that the thrust piece (20) coming from the proximal direction (101) can be fitted into the fork head (10). In this case, the projections of the thrust piece (24) latch with the supporting surfaces (181) and the thrust piece (20) is secured in the proximal direction (101) but not subjected to force. In FIG. 10, it can also be seen that the fork head (10) in the through opening (18) has a region which, at least in sections, has a larger inner diameter (182) than the core diameter of the thread (16). As a result, if the thrust piece is not yet fully latched to the fork head (10) in the final position, it is possible for the resilient cone region (27) of the thrust piece in the fork head (10) to be provided with corresponding space for spreading apart at the level of the inner diameter widening (182) for assembly with the bone anchor head region (91).

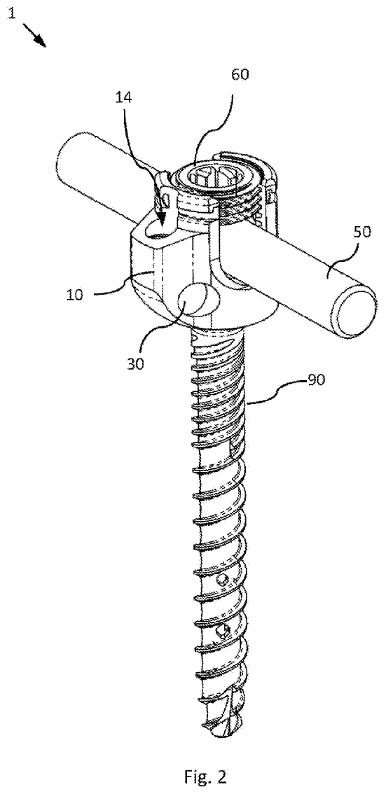

In FIG. 2 it is depicted that when the positioning element (40) is guided within an axial opening (14) and is part of the osteosynthesis device (1), and has a predetermined breaking point (45), only a part of the positioning element (40) remains in the patient after the final locking with the connecting rod (50) and the locking element (60). The same picture is obtained if the positioning element (40) is part of an instrument and is removed from the patient after the final locking with the connecting rod (50) and the locking element (60).

Alternatively, the positioning element (40) can also be provided completely as part of the osteosynthesis device (1). After the final locking with the connecting rod (50) and the locking element (60), it remains in the patient (not depicted). It is advantageous if the positioning element (40) does not protrude beyond the proximal end (101) of the fork head (10).

1. An osteosynthesis device for treating the spine, comprising a fork head which is U-shaped in a side view and which has a through opening and the fork head has two fork legs with an inner-lying thread in the proximal direction, and a connecting rod can be received therein, and a ball head receiving region is provided in the fork head in the distal direction in the through opening, and a bone anchor is pivotably mounted therein, characterized in that a transverse opening is provided on the fork head, which transverse opening communicates with the through opening of the fork head via a wall cutout, and a fixing element is mounted in this transverse opening so as to be mounted rotatably about a transverse opening axis, and the fixing element clamps the head region of the bone anchor in an angularly stable manner in the fork head with induction of a torsional moment about the transverse opening axis.

2. The osteosynthesis device according to claim 1, wherein the head region of the bone anchor in the fork head becomes movable when the torsional moment at the fixing element is unloaded.

3. The osteosynthesis device according to claim 1, wherein the centre of the inner-lying thread and the centre of the ball head receiving region define a central axis, and this central axis does not intersect with the transverse opening axis.

4. The osteosynthesis device according to claim 1, wherein the transverse opening axis is arranged approximately orthogonally and at a distance from the central axis.

5. The osteosynthesis device according to claim 1, wherein the fixing element protrudes at least in sections into the through opening of the fork head and abuts against the ball head region of the bone anchor.

6. The osteosynthesis device according to claim 1, wherein the fixing element has a radially inwardly directed contact region which abuts directly against the head region of the bone anchor, and this contact region is concavely shaped at least in sections in a side view.

7. The osteosynthesis device according to claim 1, wherein the radially inwardly directed contact region of the fixing element approximates, in a side view, at least a portion of the outer surface of the bone anchor head region.

8. The osteosynthesis device according to claim 1, wherein the radially inwardly directed contact region of the fixing element has an increased roughness, notches or teeth.

9. The osteosynthesis device according to claim 1, wherein the contact point for inducing the temporary clamping in the proximal direction lies above the equator of the bone anchor head region.

10. The osteosynthesis device according to claim 1, wherein the contact point for inducing the temporary clamping in the proximal direction lies above the centre of the ball head receiving region in the fork head.

11. The osteosynthesis device according to claim 1, wherein the fixing element is substantially pin-shaped.

12. The osteosynthesis device according to claim 1, wherein the fixing element describes a radius R2 at least at one point, and the radius R2 is greater than the radial distance between the contact region and the transverse opening axis.

13. The osteosynthesis device according to claim 1, wherein the head region of the bone anchor is defined by a radius R1 and the fixing element is defined at its thickest point by a radius R2, and the shortest distance between the central axis and the transverse opening axis is smaller than the sum of the radii R1 and R2.

14. The osteosynthesis device according to claim 1, wherein the fixing element extends beyond at least one lateral wall of the fork head and a lever is attached thereto, and an actuation of the lever causes the fixing element to rotate.

15. The osteosynthesis device according to claim 1, wherein the fork head has an axial opening, and a positioning element is guided in the axial opening, and a compression force is generated by adjusting the positioning element, which compression force is transmitted directly on the fixing element or indirectly via a lever, and a torsional moment is thereby generated on the fixing element about the transverse opening axis, which leads to the temporary clamping of the bone anchor head region in the fork head.

16. The osteosynthesis device according to claim 1, wherein a connecting rod can be inserted and can be fixed on the fork head by means of a locking element, thereby achieving an angularly stable clamping between the bone anchor head region and the fork head, and the angularly stable clamping with the aid of the locking element and the angularly stable clamping with the aid of the fixing element can be activated independently of one another and can also be combined with one another.

17. The osteosynthesis device according to claim 1, wherein the positioning element is part of the osteosynthesis device and remains in the patient after the final locking with the connecting rod and the locking element.

18. The osteosynthesis device according to claim 1, wherein the positioning element does not protrude beyond the proximal end of the fork head in the proximal direction.

19. The osteosynthesis device according to claim 1, wherein the positioning element is part of the osteosynthesis device and has a predetermined breaking point, and only a part of the positioning element remains in the patient after the final locking with the connecting rod and the locking element.

20. The osteosynthesis device according to claim 1, wherein the positioning element is part of an instrument and is removed from the patient after the final locking with the connecting rod and the locking element.

21. The osteosynthesis device according to claim 1, wherein the fixing element can assume a rotational position S1 about the transverse opening axis, in which a compression force is transmitted on the bone anchor, so that the bone anchor is held angularly stable in the ball seat, and the fixing element can assume a second rotational position S2, in which the bone anchor is held movably in the ball seat.

22. The osteosynthesis device according to claim 1, wherein the axial opening for the positioning element has an inner thread at least in sections.

23. The osteosynthesis device according to claim 1, wherein the positioning element is designed in the form of a pin and has a tool attachment point in the proximal direction.

24. The osteosynthesis device according to claim 1, wherein the positioning element is part of an instrument and the fork head has projections, openings, grooves, bars, profiles or other features which are suitable for gripping, engaging or engaging from behind an instrument, which is used to induce a tensile force on the fork head, which acts as an antagonist to the induction of a compression force via the positioning element.

25. The osteosynthesis device according to claim 1, wherein the fork head has a thrust piece, and the thrust piece has a through opening, a distally directed contact region towards the bone anchor head region, a proximally directed stave bearing, and, in the region of the bone anchor head region, a lateral opening or partial cutout, in which the fixing element is arranged in a freely movable manner.

26. The osteosynthesis device according to claim 1, wherein the fork head provides a thrust piece and, when the bone anchor head region is clamped solely by the fixing element, the thrust piece is unloaded.

27. The osteosynthesis device according to claim 25, wherein the thrust piece has open slots in the distal direction, and as a result at least three resilient arms are formed on the head receiving region and the resilient arms describe a cone on their outer side at least in sections, and the bone anchor can be fitted into the fork head coming from the distal direction.

28. The osteosynthesis device according to claim 27, wherein the fork head defines at least one inner cone section in the ball receiving region, which leads congruently to the cone of the thrust piece and, when the locking element is actuated, to the angularly stable clamping of the bone anchor head region with the fork head.

29. The osteosynthesis device according to claim 27, wherein the fork head has a region in the through opening which has, at least in sections, a larger inner diameter than the core diameter of the thread.

30. The osteosynthesis device according to claim 1, wherein the legs each provide a supporting surface with an undercut, which is effective in the proximal direction, and the thrust piece has radially outwardly directed projections at the proximal end, the projections being designed to be resilient radially inwards, so that the thrust piece, coming from the proximal direction, can be fitted into the fork head and the projections of the thrust piece latch with the supporting surfaces, and the thrust piece is secured in the proximal direction but not subjected to force.
